(19) United States
(12) Patent Application Publication
(10) Pub. No.: US 2023/0310051 A1
(43) Pub. Date: Oct. 5, 2023

(54) PATIENT SPECIFIC INSTRUMENT (PSI) METHOD & DEVICE FOR PERCUTANEOUS FIXATION OF FRACTURES

(71) Applicant: Mahmoud Alm El Din Hafez, Giza (EG)

(72) Inventors: Mahmoud Alm El Din HAFEZ, Giza (EG); Mansoor Tahnoon AL NEHAYAN, Al Ain (AE)

(21) Appl. No.: 18/021,109

(22) PCT Filed: Aug. 11, 2020
(86) PCT No.: PCT/EG2020/000020

§ 371 Date: Feb. 13, 2023

(51) Int. Cl. A61B 17/90; A61B 17/88

(52) U.S. Cl. CPC A61B 17/90; A61B 17/8861; A61B 2017/568

(57) ABSTRACT

This invention is a patient-specific surgical guide for percutaneous fixation of fractures designed for detection of the point of insertion, direction and angle of metal-ware (wires, screws, plates or nails). The patient-specific surgical guide seated on a bony landmark, the interior surface of the guide matches the surface anatomy of bones and fit in a single secure position for the reduction of bony or joint displacement and to insert wires or metal ware in bone and joints without need for fluoroscopic imaging. The guide comprising of a flying arc (3) with pointed introducers (1, 2) at its ends, detachable rods (27,28) and trajectory sleeves (6, 7, 24). The size and shape of the guide modified according to the nature of the anatomy of the percutaneous of bone and joints in these regions of displaced fractures of clavicle, proximal humerus, distal humerus, proximal radius and ulna, distal radius and ulna, hand bones, clavicle, patella distal fibula, medial malleolus, spine and feet bones.

### TECHNICAL FIELD

Using of computer assisted patient specific instruments to help percutaneous fixation of fractures and dislocations of bone and joints.

### BACKGROUND

Percutaneous fixation of fractures is usually done under x-ray (C arm) control with excessive exposure to radiation and no guidance of perfect positioning. Achieving the correct trajectory and placement of the wire for the percutaneous fixation of fractures needs to know the exact points of insertion, direction and the angle of the wire. However, the percutaneous approach has fewer complications than the open and/or dorsal approaches.

The bone anatomy and location makes percutaneous wire insertion a complicated process. The percutaneous fixation of fractures has a long learning curve, furthermore the patient has exposed to prolonged radiographic exposure in the time that the surgeon tries to guide the wire to the correct position.

The challenge of the percutaneous fixation of fractures is to reduce the amount of radiation exposure for both the patient and the surgeon.

More complications were founded in using the technique of percutaneous fixation of fractures in the cases of acetabular fractures, proximal humours fractures, scaphoid fractures due to the bone geometry, location, degree of comminution, and bone quality. Nevertheless, percutaneous fixation of fractures is a highly recommended in many cases of fractures than the other techniques like plate fixation, joint replacement and/or intramedullary nailing.

### SUMMARY

In this invention, the technique of the computer assisted surgery is used to build and design a patient specific surgical guide for percutaneous fixation of fractures. It aims to get an accurate positioning of wires or implants in bone through percutaneous applications. The technique based on the preoperative planning to observe the exact points of insertion, direction and the angle of the wire and/or screws. Percutaneous fixation of fractures and dislocations is applicable to any bone and joints that palpable and easily detectable through the skin. By using of surface anatomy, it is possible to identify custom bony landmarks, these rigid fixed structure are seen in CT scan and planning of the surgery can be done preoperatively based on these landmarks. When one or more of these landmarks are broken and displaced, other nearby landmarks are used to guide the reduction.

A 3D model of the bone was created on the 3D modelling software based on the 2D images captured from the X-ray, CT-scan and Ultrasound. The 2D images of the both the bony land marks and skin collected by a potable 3D scanner with mechanical touch probe and saved in Dicom format. The CT-scan collect the data of the bone (hard tissues) and the ultrasound imaging collect the data of the skin and soft tissues. At this moment, we have a complete view and a complete data for the skin, soft tissues and bone landmarks (e.g. styloid process of radius, ulna head, olecranon, spinous process, tibial tuberosity, lateral malleolus, medial malleolus, fibula head, . . . ). These data will be inserted to the image processing software to create the 3D model of the bone.

The second phase of this technique is to design the patient specific surgical guide for percutaneous fixation of fractures. This guide allows the surgeons to detect the best and accurate insertion point and direction of the wires as well the surgery will be minimally invasive.

The guide will be used as a patient-specific surgical guide for percutaneous fixation of fractures, dislocation and osteotomies of bony structures that are palpable under the skin, the guide designed for detection of the point of insertion and trajectory of metal-ware (wires, screws, plates or nails). The guide consisting of different parts, that will be assembled together in different shapes according to according to the location and orientation of the fractures of like: clavicle, proximal humerus, distal humerus, proximal radius and ulna, distal radius and ulna, hand bones, clavicle, patella, distal fibula, medial malleolus, spine or feet bones. The guide produced from a radiolucent material and it does not interfere with imaging if needed. The guide should be rigid to allow manipulation without bending. The manufacturing material is Nylon P12 or Nylon P11.

### DRAWINGS DESCRIPTION

FIG. 1-A: A top view of the patient specific surgical guide for the fixation of the distal radius fracture over the skin. The figure shows the medial seating introducers ((1) and (4)), the lateral seating introducers ((2) and (5)), the reduction arc (3), the medial detachable accessory rod (27), the lateral detachable accessory rod (28), secondary fastening hole (8), the primary percutaneous wire guiding sleeve (6), the secondary percutaneous wire guiding sleeve (7) and the bridge (9).

FIG. 1-B: An Isometric view of the patient specific surgical guide for the fixation of the distal radius fracture over the skin. The figure shows the medial pointed introducer (1), the reduction arc (3), the medial detachable accessory rod (27), the primary percutaneous wire guiding sleeve (6), the secondary percutaneous wire guiding sleeve (7) and primary fastening hole (10).

FIG. 2-A: An Isometric view of the patient specific surgical guide for the fixation of the distal radius fracture. The figure the medial seating introducers ((1) and (4)), the lateral seating introducers ((2) and (5)), the reduction arc (3), the medial detachable accessory rod (27), the lateral detachable accessory rod (2), the primary percutaneous wire guiding sleeve (6), the secondary percutaneous wire guiding sleeve (7), the bridge (9), primary fastening hole (10) and the internal seating surface of the seating introducers (19) and assembly holes (29).

FIG. 2-B: A top view of the patient specific surgical guide for the fixation of the distal radius fracture showing its position over the bone. The figure shows the medial seating introducers ((1) and (4)), the lateral seating introducers ((2) and (5)), the reduction arc (3), the medial detachable accessory rod (27), the lateral detachable accessory rod (28), secondary fastening hole (8), the primary percutaneous wire guiding sleeve (6), the secondary percutaneous wire guiding sleeve (7), the distal radius fracture (13), radius (11) and ulna (12).

FIG. 3-A: A top view of the patient specific surgical guide for the fixation of the distal radius fracture in its original position. The figure shows the reduction indicator (15) between the two parts of the reduction arc (3), and the guiding pin (14).

FIG. 3-B: A top view of the patient specific surgical guide for the fixation of the distal radius fracture in its final position (16) after bone reduction and the fixation of the fracture.

FIG. 4-A: A front view of the patient specific surgical guide for the fixation of the fibula fracture over the skin. The figure shows the medial seating introducers ((1) and (4)), the lateral seating introducers (2), the reduction arc (3), the lateral detachable accessory rod (28), the primary percutaneous wire guiding sleeve (6).

FIG. 4-B: An Isometric view of the patient specific surgical guide for the fixation of the fibula fracture over the skin. The figure shows the lateral seating introducers ((2) and (5)), the reduction arc (3), the primary percutaneous wire guiding sleeve (6) and primary fastening hole (10).

FIG. 5-A: An Isometric view of the patient specific surgical guide for the fixation of the fibula fracture over the bone. The figure shows the lateral seating introducers ((2) and (5)), the lateral detachable accessory rod (28), the primary percutaneous wire guiding sleeve (6), fibula (17) and tibia (18).

FIG. 5-B: A front view of the patient specific surgical guide for the fixation of the fibula fracture over the bone. The figure shows the medial seating introducers (1), the lateral seating introducers ((2) and (5)), the reduction arc (3), the primary percutaneous wire guiding sleeve (6), fibula (17), tibia (18) and fibula fracture (21).

FIG. 6-A: An Isometric view of the patient specific surgical guide for the fixation of the fibula fracture. The figure shows the medial seating introducers (1), the lateral seating introducers ((2) and (5)), the lateral detachable accessory rod (28), the primary percutaneous wire guiding sleeve (6), primary fastening hole (10), secondary fastening hole (8), the lateral detachable accessory rod (28), the final position of the reduction indicator (16) and the internal seating surface of the pointed introducer (19).

FIG. 6-B: An Isometric view of the reduction joint. The figure shows guiding pin (14), guiding hole (20) and assembly holes (29).

FIG. 7-A: A front view of the patient specific surgical guide for the fixation of the medial malleolus fracture over the skin. The figure shows the medial pointed introducer (1), the lateral seating introducers ((2) and (4)), the reduction arc (3), the medial detachable accessory rod (27) and the primary percutaneous wire guiding sleeve (6).

FIG. 7-B: A front view of the patient specific surgical guide for the fixation of the medial malleolus fracture over the bone. The figure shows the medial seating introducers (1), the lateral seating introducers ((2) and (4)), the medial detachable accessory rod (27), the primary percutaneous wire guiding sleeve (6), fibula (17), tibia (18) and the medial malleolus fracture (22).

FIG. 8-A: A lateral view of the patient specific surgical guide for the fixation of the medial malleolus fracture over the bone. The figure shows the medial detachable accessory rod (27), primary fastening hole (10) and fibula (17).

FIG. 8-B: A medial view of the patient specific surgical guide for the fixation of the medial malleolus fracture over the bone. The figure shows the medial seating introducers (1), the medial detachable accessory rod (27), primary fastening hole (10), tibia (18) and the medial malleolus fracture (22).

FIG. 9: An isometric view of the patient specific surgical guide for the fixation of the medial malleolus fracture over the bone in its original position. The figure shows the reduction indicator (15) between the two parts of the reduction arc (3).

FIG. 10-A: An Isometric view of the patient specific surgical guide for the fixation of the medial malleolus fracture. The figure shows the medial seating introducers ((1) and (4)), the lateral seating introducers (2), the medial detachable accessory rod (27), the primary percutaneous wire guiding sleeve (6), primary fastening hole (10) and the internal seating surface of the pointed introducer (19).

FIG. 10-B: A front view of the patient specific surgical guide for the fixation of the medial malleolus fracture. The figure shows the medial seating introducers ((1) and (4)), the lateral seating introducers (2) and the primary percutaneous wire guiding sleeve (6).

FIG. 11: A posterior view of the patient specific surgical guide for the fixation of the distal humerus fracture over the bone. The figure shows the medial seating introducers ((1) and (4)), the lateral seating introducers (2), the medial detachable accessory rod (27), the medial primary percutaneous wire guiding sleeve (7), the lateral primary percutaneous wire guiding sleeve (6), lateral secondary percutaneous wire guiding sleeve (24), secondary fastening hole (8), humerus (25), olecranon (26), ulna (12) and the distal humerus fracture (23).

FIG. 12-A: An isometric view of the patient specific surgical guide for the fixation of the distal humerus fracture over the bone. The figure shows the medial primary percutaneous wire guiding sleeve (7), the lateral primary percutaneous wire guiding sleeve (6) and the lateral secondary percutaneous wire guiding sleeve (24), primary fastening hole (10) and secondary fastening hole (8).

FIG. 12-B: An isometric view of the patient specific surgical guide for the fixation of the distal humerus fracture in its original position. The figure shows the reduction indicator (15) and primary fastening hole (10).

FIG. 13: A front view of the patient specific surgical guide for the fixation of the distal humerus fracture in its original position. The figure shows the reduction indicator (15), the medial primary percutaneous wire guiding sleeve (7), the lateral primary percutaneous wire guiding sleeve (6) and lateral secondary percutaneous wire guiding sleeve (24).

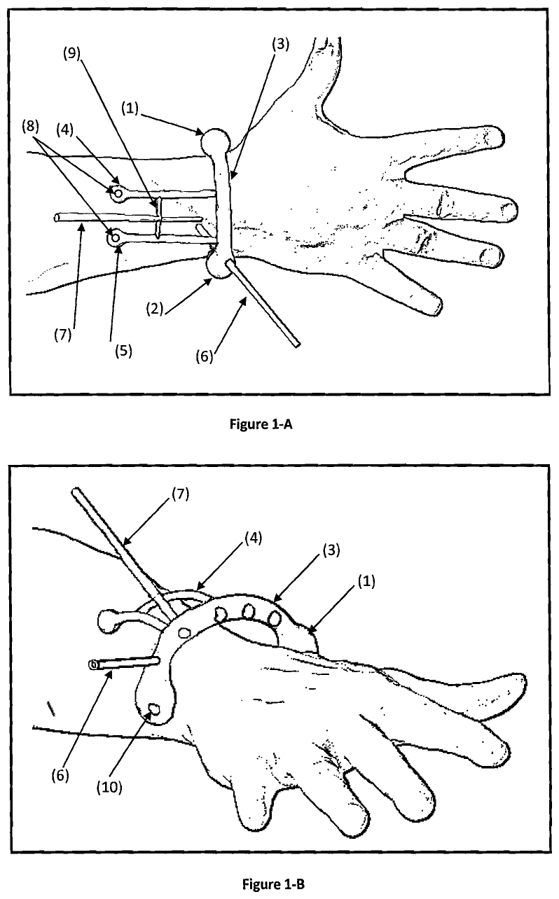

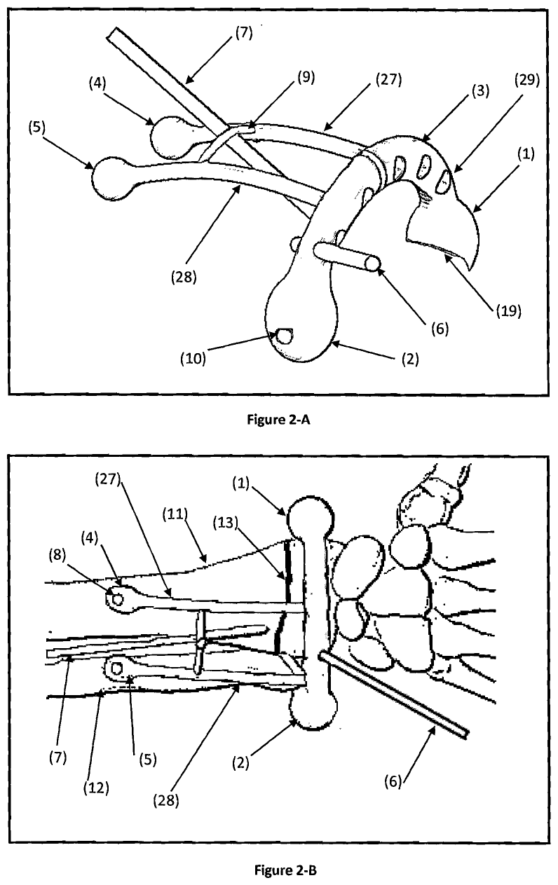

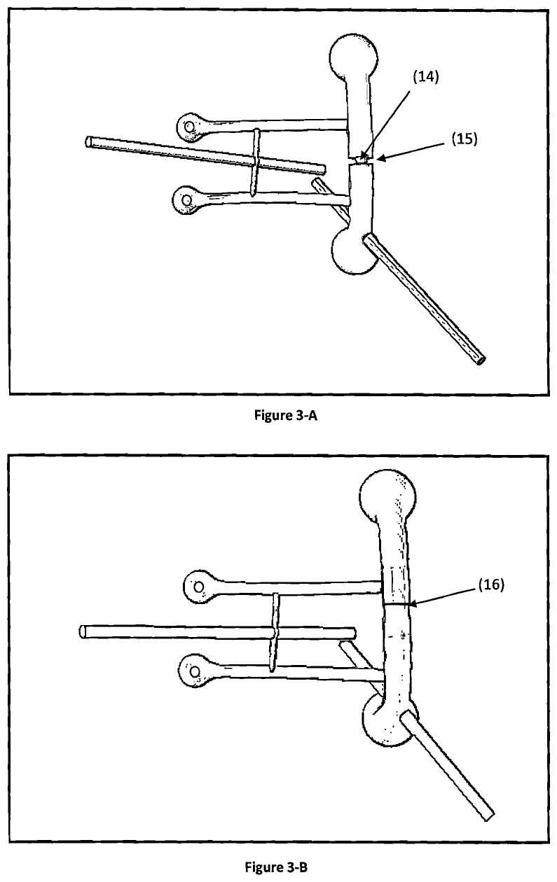

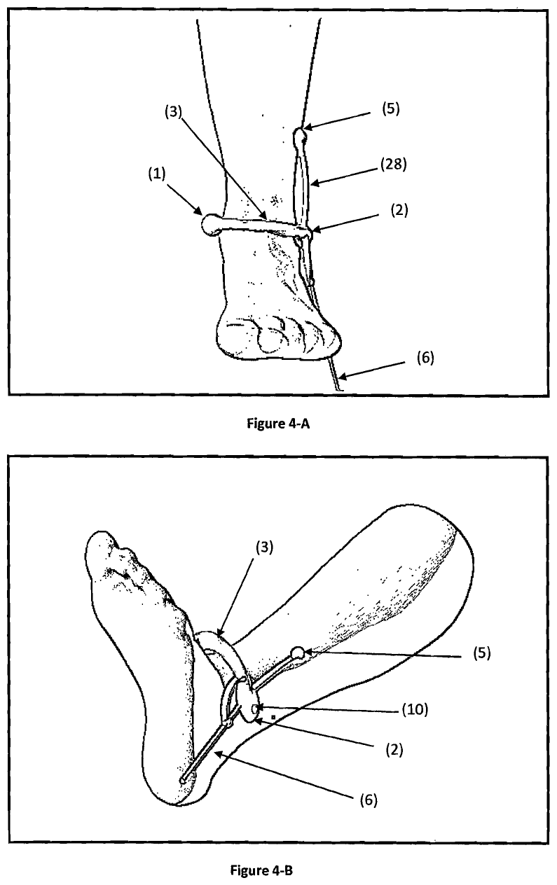

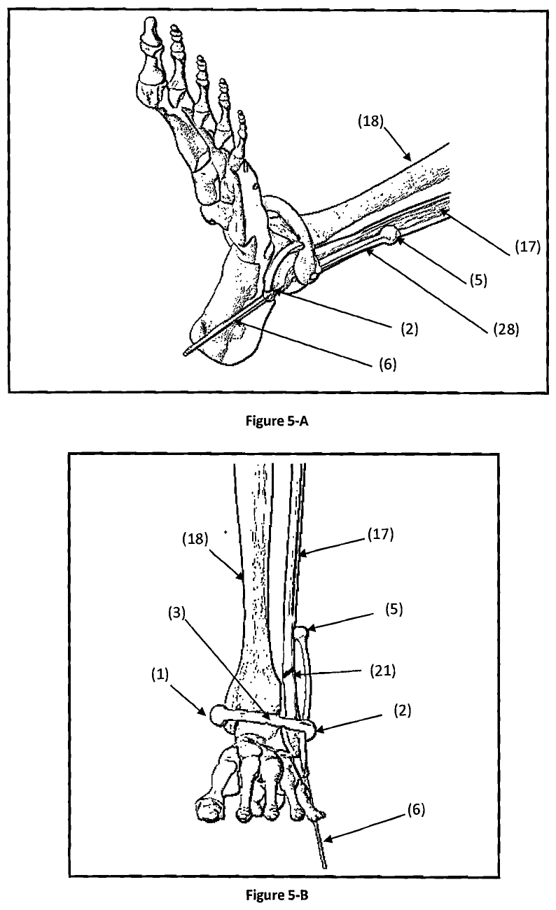

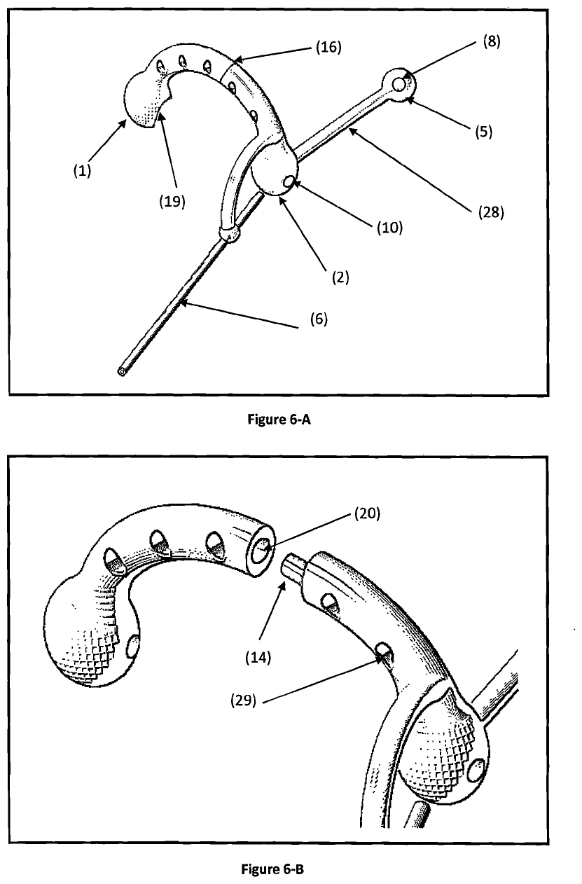

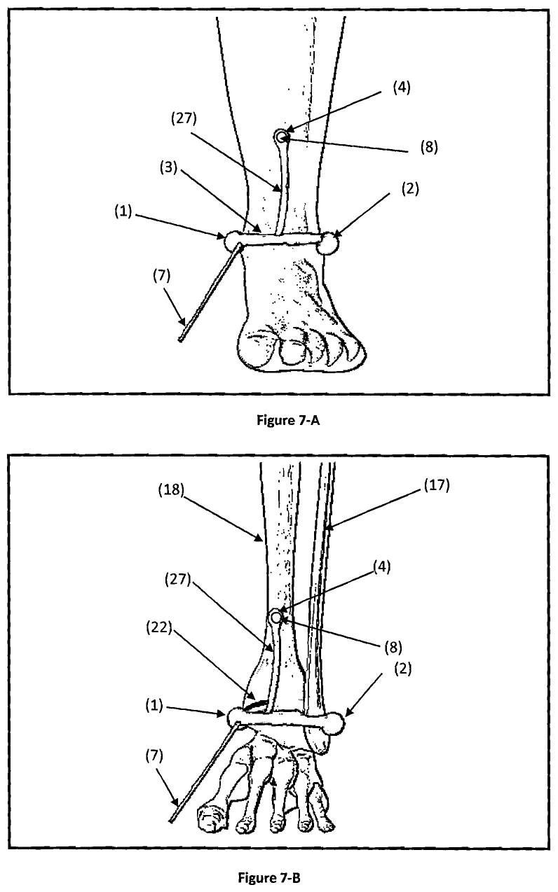

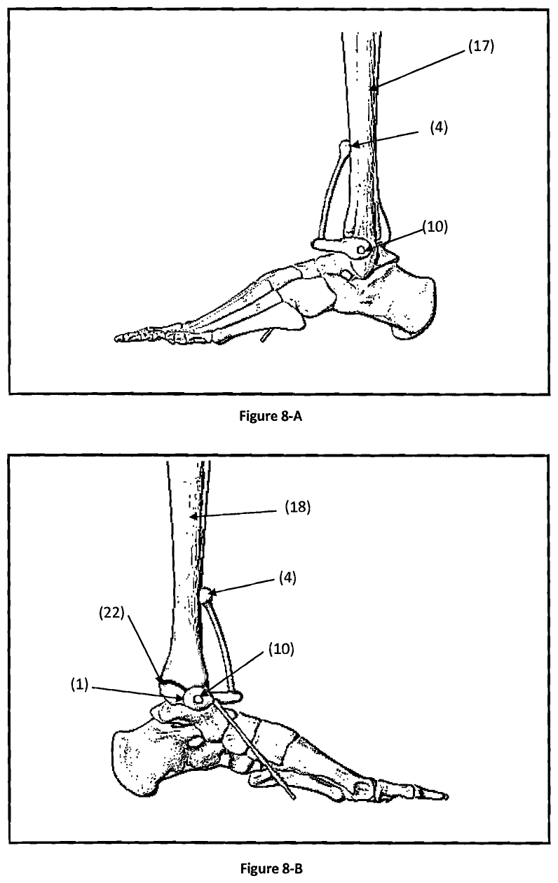

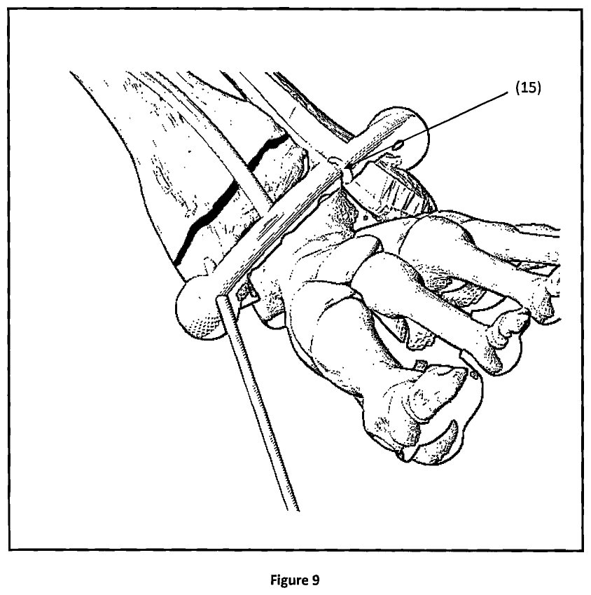

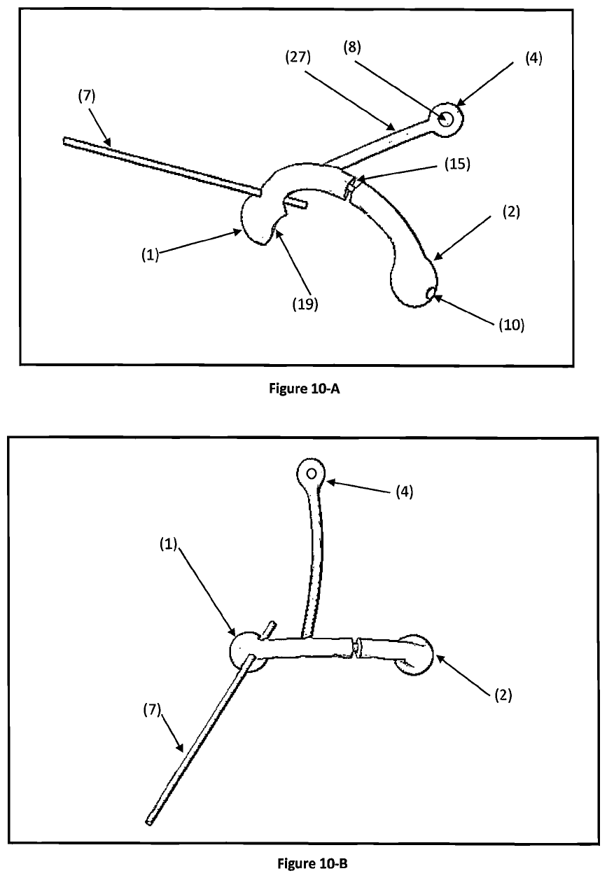

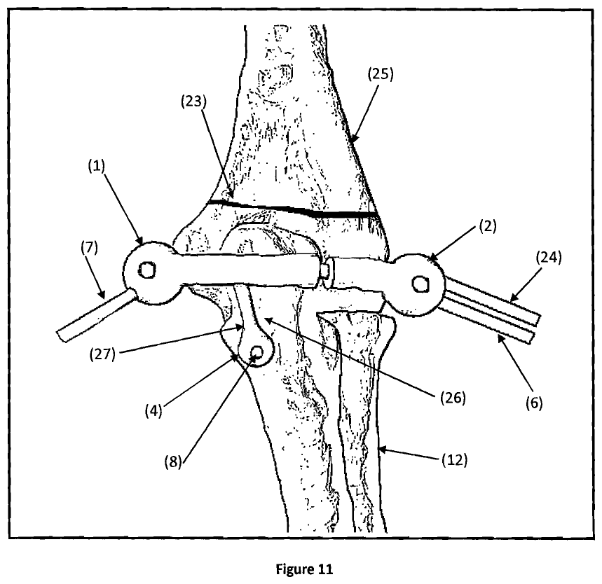

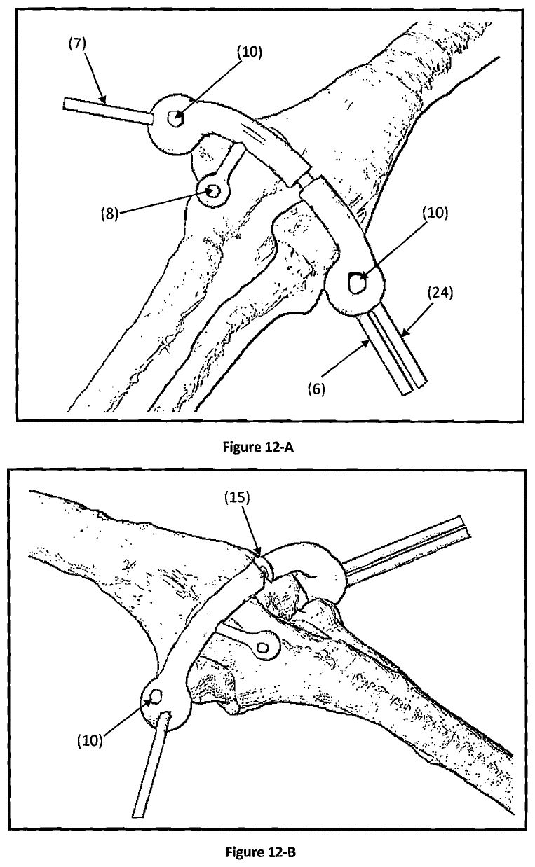

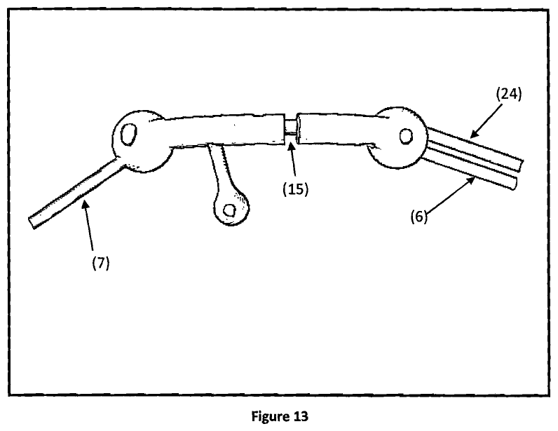

### DETAILED DESCRIPTION

The guide comprising of a reduction arc (3), detachable accessory rods [(27) and (28)] and percutaneous wire guiding sleeves [(6), (7) and (24)]. The reduction arc has a several holes in its body (29), these holes are an assembly holes. The detachable accessory rods seating introducers [(27) and (28)] are attached and de-attached to the reduction arc by inserting the circular ends of these rods inside the assembly holes in reduction arc body.

The reduction arc has a two spherical ends which are [(1) and (2)], the function of these introducers is to seat over the skin on the bony landmarks in a single and secure position to fix the guide over the bone. The internal surface (19) of these seating introducers have the same anatomy shape like the bony landmark, which is mean it will fit in its position over the bone.

The lateral pointed introducer (1) seated on the lateral bone landmark, while the medial pointed introducer (2) seated on the medial bone landmark.

Also, the detachable accessory rods [(27) and (28)] have seating introducers [(4) and (5)] at its ends. The function seating introducers [(4) and (5)] is same as the seating introducers which are located at the ends of the reduction arc. It seated on the bony landmarks on the damaged bone. the detachable accessory rods [(27) and (28)] provide an additional stability to the guide over the bone. The medial detachable accessory rod (27) seated on the medial bone landmarks like the ulna or fibula; while the lateral detachable accessory rod (28) seated on the lateral bone landmarks like the radius, tibia or olecranon. That means, the patient-specific surgical guide seated on a bony landmark wherein the internal surface (19) of the seating introducers [(1), (2), (4) and (5)] matches the surface anatomy of bones and fit in a single secure position for the reduction of bone or joint displacement and to insert wires or metal ware in bone and joints without need for fluoroscopic imaging.

In case of lateral fractures like distal fibula and distal ulna fractures, the surgeon attached only the lateral detachable accessory rod (28) to the reduction arc (3), there is no need to use the medial one (27). And vice versa, In case of medial fractures like distal humerus and medial malleolus fractures, the surgeon attached only the medial detachable accessory rod (27) to the reduction arc (3), there is no need to use the lateral one (28).

In case of the extendable fractures like the distal radius fracture, the surgeon should be attached the lateral and the medial detachable accessory rod (28) and (27) to the reduction arc (3) to secure the position of the guide over the bone.

For the distal radius fracture, the medial and the lateral detachable accessory rods ((27) and (28)) assemble to the reduction arc and seated on the radius bone and ulna bone respectively. Wherein in the distal humerus fracture, the medial detachable accessory rod (27) assembled to the reduction arc and seated on the olecranon bone. In the medial malleolus fracture, the medial detachable accessory rod (27) assembled to the reduction arc and seated on the tibia bone. In case of the fibula fracture, the lateral detachable accessory rod (28) assembled to the reduction arc and seated on the fibula bone. In the case of pedicle screw fixation for spinal fractures fixation or fusion or other spinal surgery, the surgeon assembles the medial and the lateral detachable accessory rods ((27) and (28)) to the reduction arc and seated on at least two of the percutaneous landmarks like spinous processes and transverse processes.

The seating introducers [(1) and (2)] have a primary fastening holes (10) and the seating introducers [(4) and (5)] have a secondary fastening holes (8). These fastening holes used for fixing the guide over the bone by using of surgical pins and/or wire. This process will give the guide an additional stability and rigidity over the bone and allows the surgeon to insert the percutaneous wires to fix the fractures easily, especially for the low-experience surgeons who didn't have experience with this kind of surgeries.

The guide has percutaneous wire guiding sleeves [(6), (7) and (24)]. All of these sleeves are cannulated to directed the wires through the internal hole in the percutaneous wire guiding sleeve to determine and detection the point of insertion and trajectory of metal-ware (wires, screws, plates or nails) in the bone to fix the displaced fractures. These sleeves are located medially and/or laterally to fix the fractures of distal radius, distal humerus, fibula fractures and medial malleolus fractures from both of the medial and lateral sides.

According to the orientation and location of the fracture, the surgeon has the ability to use one or more of these sleeve as follows:
1. In the case of distal radius fracture, the surgeon will use the design of the guide with the medial primary percutaneous wire guiding sleeve (7) which is located in the medial side and lateral primary percutaneous wire guiding sleeve (6) which located in the lateral side to fix the radius fracture.
2. In the case of the distal humerus fracture, the surgeon will use the design of the guide with two primary percutaneous wire guiding sleeves [(7) and (6)] which are located in both medial and lateral sides respectively, and secondary percutaneous wire guiding sleeve (24) which located in the lateral side.
3. In the case of the medial malleolus fracture, the surgeon will use the design of the guide with only one medial primary percutaneous wire guiding sleeve (7) which is located in the medial side.
4. In the case of the fibula fracture, the surgeon will use the design of the guide with only one lateral primary percutaneous wire guiding sleeve (6) which is located in the lateral side.
5. In the case of spinal fractures, the surgeon will use the design of the guide with the medial primary percutaneous wire guiding sleeve (7) which is located in the medial side and lateral primary percutaneous wire guiding sleeve (6) which located in the lateral side.

The reduction arc (3) consisting of two parts with a gap between the parts, the gap introduces the reduction indicator (15); when the surgeon inserts the percutaneous wire through the cannulated percutaneous wire guiding sleeve to fix the fracture, the two parts of the reduction arc will move to each other and when the two parts meet together at the final position (16), it means the displaced fracture is reduced.

The seating introducers [(1), (2), (4) and (5)] detect the positions of the fiducial markers that were inserted on the bone preoperatively, these markers are additional landmarks during surgery for a certain structures of bone when the landmarks are hidden due to opacity. While, the percutaneous wire guiding sleeves directed the percutaneous pins to make some hidden landmarks visible or prominent.

1. A patient-specific surgical guide for percutaneous fixation of fractures, dislocation and osteotomies of bony structures that are palpable under the skin, the guide designed for detection of the point of insertion and trajectory of metal-ware including wires, screws, plates or nails, comprising a flying arc (3) with pointed introducers [(1) and (2)] at its ends, detachable accessory rods at medial (27) and lateral (28) sides with pointed introducers [(4) and (5)], and trajectory sleeves [(6), (7) and (24)].

2. The patient-specific surgical guide according to claim 1 seated on a bony landmark wherein the internal surface (19) of the pointed introducers [(1), (2), (4) and (5)] matches the surface anatomy of bones and fit in a single secure position for the reduction of bone or joint displacement and to insert wires or metal ware in bone and joints without need for fluoroscopic imaging.

3. The patient-specific surgical guide according to claim 1, the shape of the flying arc (3), detachable accessory rods [(4) and (5)] and trajectory sleeves [(6), (7) and (24)] is modified according to the percutaneous procedures on bone and joints in undisplaced and displaced fractures of clavicle, proximal humerus, distal humerus, proximal radius and ulna, distal radius and ulna, hand bones, clavicle, patella, distal fibula, medial malleolus, spine and feet bones.

4. The patient-specific surgical guide according to claim 1, the guide is designed based on the 2D images captured from one of an X-ray, CT-scan and Ultrasound.

5. The patient-specific surgical guide according to claim 1, the guide is an image based for both the bony land marks and skin.

6. The patient-specific surgical guide according to claim 1, the guide is designed based on collecting the data of the surface shape of the bony and soft tissue landmarks by using a portable 3D scanner with a mechanical touch probe.

7. The patient-specific surgical guide according to claim 1, the guide comprises pointed introducers ((1), (2), (4) and (5)) on each end of the flying arc (3) and at the ends of the two detachable accessory rods ((27) and (28)) to be seated percutaneously or subcutaneously on bony landmark in any region of the body that has a surface anatomy including shoulder and scapula, elbow joint, carpal and hand joints, pelvic, distal femoral trochanteric region, distal femur, proximal tibia, or ankle joint.

8. The patient-specific surgical guide according to claim 7, the internal surface (19) of the pointed introducers ((1), (2), (4) and (5)) matches the bone landmarks anatomy at the area that will be seated on it.

9. The patient-specific surgical guide according to claim 7, the pointed introducers ((1), (2), (4) and (5)) have fastening holes [(8) and (10)] on its body to allow and secure the fixation of the patient-specific surgical guide over the skin in the bony landmarks.

10. The patient-specific surgical guide according to claim 1, the flying arc (3) comprises two parts with a gap between each other, the gap introduces the reduction indicator (15), and when the two parts meet together (16), the displaced fracture is reduced.

11. The patient-specific surgical guide according to claim 1, the guide comprises detachable accessory rods ((27) and (28)) are attached and de-attached to the flying arc (3) through the assembly holes (29) in its body.

12. The patient-specific surgical guide according to claim 1, the medial detachable accessory rod (27) designed to seat on medial bony landmarks on the radius, tibia or olecranon.

13. The patient-specific surgical guide according to claim 1, the lateral detachable accessory rod (28) designed to seat on medial bony landmarks on the ulna or fibula.

14. The patient-specific surgical guide according to claim 1, the trajectory sleeves ((6), (7) and (24)) are cannulated to direct the wires through the internal hole in the trajectory sleeve to determine the required trajectory in the bone to fix the displaced fractures.

15. The patient-specific surgical guide according to claim 1, the guide comprises trajectory sleeves ((6), (7) and (24)), that are located medially and/or laterally to fix the fractures of distal radius, distal humerus, fibula fractures and medial malleolus fractures from both of the medial and lateral sides.

16. The patient-specific surgical guide according to claim 1, the manufacturing material of the guide is radiolucent and does not interfere with imaging, and the guide is rigid to allow manipulation without bending.

17. The patient-specific surgical guide according to claim 1, the guide comprises a design that allows assembly of the detachable accessory rods ((27) and (28)) to the flying arc (3) medially or laterally according to the orientation and location of the displaced fracture to provide additional stability to the guide over the bone; and
in the distal radius fracture, the medial and the lateral detachable accessory rods ((27) and (28)) assemble to the flying arc and seated on the radius bone and ulna bone respectively,
in the distal humerus fracture, the medial detachable accessory rod (27) assembled to the flying arc and seated on the olecranon bone,
in the medial malleolus fracture, the medial detachable accessory rod (27) assembled to the flying arc and seated on the tibia bone, and
in the fibula fracture, the lateral detachable accessory rod (28) assembled to the flying arc and seated on the fibula bone.

18. The patient-specific surgical guide according to claim 1, the guide comprises primary and secondary trajectory sleeves ((6), (7) and (24)) which are located medially and/or laterally to direct the wires to fix the fractures according to the orientation and location of the displaced fractures; and
in the distal radius fracture, there is a medial primary trajectory sleeve (7) located in the medial side and lateral primary trajectory sleeve (6) located in the lateral side,
in the distal humerus fracture, there are two primary trajectory sleeves [(7) and (6)] located in both medial and lateral sides respectively, and the secondary trajectory sleeve (24) located in the lateral side,
in the medial malleolus fracture, there is one medial primary trajectory sleeve (7) located in the medial side, and
in fibula fracture, there is one lateral primary trajectory sleeve (6) located in the lateral side.

19. The patient-specific surgical guide according to claim 1, the guide comprises pointed introducers [(1), (2), (4) and (5)] which detect the positions of the fiducial markers inserted on the bone preoperatively, the markers are additional landmarks during surgery for certain structures of bone when the landmarks are hidden due to opacity.

20. The patient-specific surgical guide according to claim 1, the trajectory sleeves direct the percutaneous pins to make hidden landmarks visible or prominent.
